(19) United States
(12) Patent Application Publication
(10) Pub. No.: US 2026/0351393 A1
(43) Pub. Date: Nov. 5, 2026

(54) IN-ROOM AIR-QUALITY SENSOR SYSTEM FOR BOWEL MOVEMENT DETECTION AND CAREGIVER NOTIFICATION

(71) Applicant: Brigham Young University, Provo, UT (US)

(72) Inventor: Robert B. SOWBY, American Fork, UT (US)

(73) Assignee: Brigham Young University, Provo, UT (US)

(21) Appl. No.: 19/650,034

(22) Filed: Apr. 16, 2026

Related U.S. Application Data
(60) Provisional application No. 63/789,785, filed on Apr. 16, 2025.

(51) Int. Cl. G16H 40/20; G01N 33/00; G08B 21/18

(52) U.S. Cl. CPC G16H 40/20; G01N 33/0063; G01N 33/0044; G01N 33/0047; G01N 33/005; G08B 21/182

(57) ABSTRACT

A non-contact bowel movement detection system for a dependent individual includes a patient unit, a cloud-based processing platform, and a mobile application. The patient unit is positioned in an occupied room and includes a housing with air intake apertures on a side surface and air exhaust apertures on a top surface. The housing contains a microcontroller with wireless communication, gas sensors including hydrogen, hydrogen sulfide, and volatile organic compounds, and a temperature and humidity sensor. An internal fan draws ambient air through the intake apertures, across the sensors, and out through the exhaust apertures to enhance detection. The microcontroller periodically collects sensor data and transmits it to the cloud platform. The cloud platform applies a machine-learning model to determine a bowel movement probability and generates an alert when a threshold is met. The mobile application receives and forwards the alert to a caregiver device.

### PRIORITY CLAIM

This application claims the benefit of and priority to U.S. Provisional Patent Application 63/789.785 filed on Apr. 16, 2025.

### TECHNICAL FIELD

The present disclosure relates generally to environmental sensing systems and, more particularly, to an in-room air-quality sensor system that detects bowel movements in a dependent individual by analyzing ambient air and alerts a caregiver via a mobile application.

### BACKGROUND

Timely cleanup of a bowel movement is essential for the health of a dependent individual, the sanitary condition of the surroundings, and the convenience of the caregiver. Dependent individuals—such as infants, disabled children, and adults in care centers or hospitals—may not know or be able to communicate that they have had a bowel movement and need attention. When bowel movements are not immediately cleaned up (e.g., overnight), the affected individual may experience discomfort, skin rashes, urinary tract infections, and other complications, while caregivers may have to deal with unsanitary spills, consume extra diapers and wipes, and wash contaminated clothing and bedding.

Existing approaches to detecting bowel movements in dependent individuals typically require direct physical contact with the individual or direct inspection by a caregiver. Wearable sensors embedded in diapers, for example, require physical attachment to the individual and may cause discomfort, require frequent replacement, or fail to function reliably. Scheduled caregiver checks, particularly during overnight care, may result in prolonged exposure of the individual to fecal material. These limitations create a need for a passive, contactless, continuous monitoring system that can reliably detect a bowel movement from a distance and promptly notify the caregiver.

Human feces produce a characteristic mixture of volatile organic compounds (VOCs) and gases, including hydrogen (H2), hydrogen sulfide (H2S), and various volatile organic compounds. These chemical signatures are detectable at low concentrations in ambient room air. Advances in low-cost microelectromechanical systems (MEMS) gas sensors, Wi-Fi-enabled microcontrollers, cloud computing, and machine learning have made it feasible to build a compact, affordable, continuously operating detection system that distinguishes the chemical signature of a bowel movement from baseline room air quality.

A need exists for a passive, non-contact bowel movement detection system that monitors room air quality continuously, processes sensor data to distinguish bowel movement events from normal ambient conditions, and delivers timely alerts to a caregiver's mobile device, without requiring the dependent individual to perform any action or wear any device.

### SUMMARY OF THE DISCLOSURE

Disclosed herein is an in-room air-quality sensor system for bowel movement detection and caregiver notification

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. The advantages of the disclosure will become better understood with regard to the following description and accompanying drawings where:

FIG. 1 illustrates a system overview diagram of the in-room bowel movement detection system, showing the patient unit, cloud processing platform, and mobile application and their interconnections.

FIG. 2 illustrates an exploded view of the patient unit, showing the housing lid, fan, printed circuit board with sensors, and housing base.

FIG. 3 illustrates a top view of the printed circuit board (PCB) showing the layout of the microcontroller, antenna, and four sensor components.

FIG. 4 illustrates a flowchart of the event detection algorithm, showing the steps of sensor sampling, filtering, neural network inference, rolling average computation, threshold comparison, and caregiver notification.

FIG. 5 illustrates a detailed data flow diagram of the sensor signal processing pipeline, showing the five-channel sensor inputs (H2, H2S, VOC, Temp, Humidity) through on-device filtering and normalization, artificial neural network (ANN) inference to compute bowel movement probability P, rolling average computation, threshold detection, and push notification dispatch to the caregiver's mobile application.

FIG. 6 illustrates a deployment diagram showing an example installation of the patient unit in a bedroom near a dependent individual's bed, including the detection range, wireless communication path to the cloud processing platform, and caregiver alert delivery to a mobile device.

### DETAILED DESCRIPTION

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or similar parts. It is further noted that elements disclosed with respect to particular embodiments are not restricted to only those embodiments in which they are described. For example, an element described in reference to one embodiment or figure may be alternatively included in another embodiment or figure, regardless of whether or not those elements are shown or described in another embodiment or figure. In other words, elements in the figures may be interchangeable between various embodiments disclosed herein, whether shown or not.

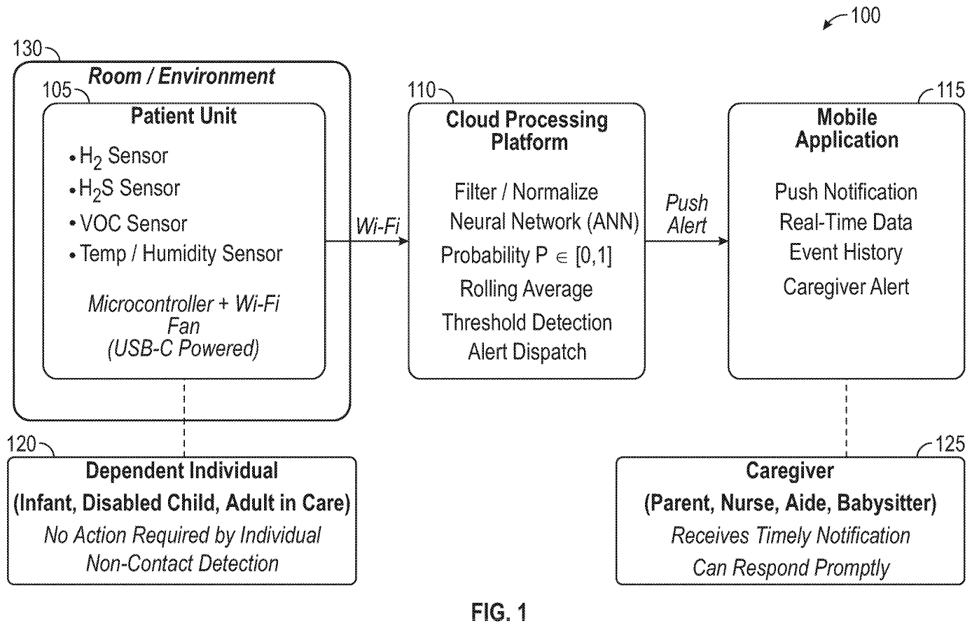

FIG. 1 illustrates a system overview of an in-room bowel movement detection system 100. System 100 may include a patient unit 105, a cloud processing platform 110, and a mobile application 115. Patient unit 105 may be positioned in a room or environment occupied by a dependent individual 120, such as an infant (I), a disabled child (DC), or an adult in care (AC), and may sample ambient air in the room continuously without requiring any action (AR) by the individual. Patient unit 105 may transmit sensor data to cloud processing platform 110 via a Wi-Fi connection 130. Cloud processing platform 110 may filter and normalize the received sensor data, apply a neural network (ANN) to compute a bowel movement probability P in the range [0, 1], compute a rolling average of probability values, and perform threshold detection to determine whether a bowel movement has occurred. When the rolling average exceeds a predetermined threshold, cloud processing platform 110 may dispatch an alert notification to mobile application 115 installed on a caregiver's (CA) mobile device 125. Mobile application 115 may display the alert to the caregiver's mobile device 125, enabling the caregiver to respond promptly.

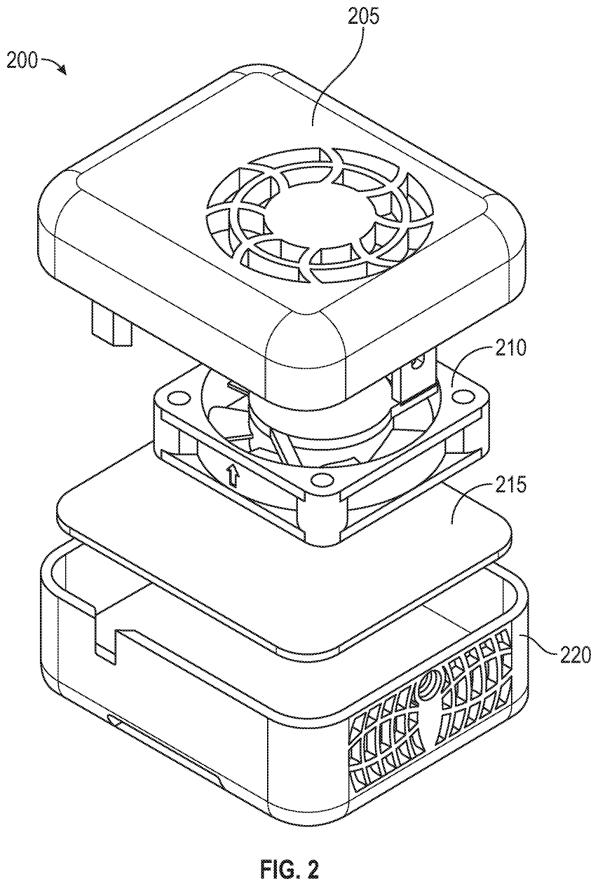

Patient unit 105 may include a housing 220 (see FIG. 2), a printed circuit board (PCB) 215, a fan 210, a microcontroller 305 (U1), and a plurality of sensors. The plurality of sensors may include a hydrogen gas (H2) sensor 310 (U2) measures a concentration of hydrogen gas in ambient air, a hydrogen sulfide gas (H2S) sensor 315 (U3) that measures a concentration of hydrogen sulfide gas in ambient air, a volatile organic compound (VOC) sensor 320 (U4) that measures concentrations of volatile organic compounds in ambient air, and a combined temperature and humidity (H/T) sensor 330 (U5) that measures ambient temperature and relative humidity. Fan 210 may draw ambient air from outside housing 220 through air intake apertures in the sides of housing 220, across the sensors on PCB 215, and expel the air through exhaust apertures in the top of housing 220, increasing the effective detection range of patient unit 105.

Housing 220 may be a compact, approximately rectangular enclosure measuring approximately 3.5 cm wide by 4.5 cm deep by 6.5 cm tall, suitable for placement on a floor, table, or other surface near a dependent individual's bed, or for mounting on a bed post using a Velcro strap or on a wall using nail holes on the rear panel of housing 220. Housing 220 may be formed from a 3D-printed plastic material, or alternatively from injection-molded plastic for volume manufacturing. Patient unit 105 may be powered via a USB-C power connector 335 on housing 220.

FIG. 2 illustrates an exploded view of patient unit 105. Patient unit 105 200 may include a housing lid 205, a fan 210, a PCB 215, and a housing base 220. PCB 215 may have microcontroller 305 (U1) mounted thereon. Microcontroller 305 may be an ESP3253 microcontroller that includes a Wi-Fi antenna 350 for wireless communication. PCB 215 may further have H2 sensor 310 (U2), H2S sensor 315 (U3), VOC sensor 320 (U4), and temperature/humidity sensor 330 (U5) mounted thereon. Fan 210 may be mounted above PCB 215 and may draw air across the sensors. Housing base 220 may include air intake apertures on its sides and mounting features on its rear surface. Housing lid 205 may include air exhaust apertures on its top surface through which air drawn by fan 210 is expelled.

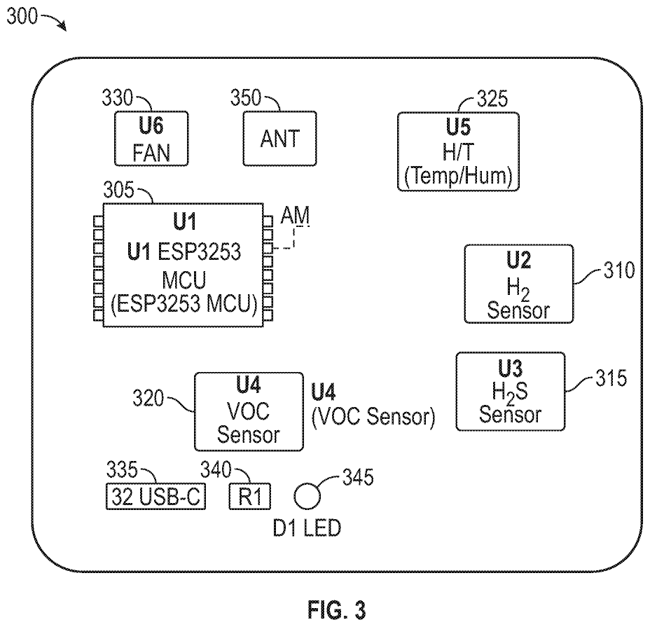

FIG. 3 illustrates a top view of PCB 300 as depicted in FIG. 2 as 215. Microcontroller 305 (U1, ESP3253 MCU) may be centrally positioned on PCB 300. H2 sensor 310 (U2) may be positioned on PCB 215 and may measure hydrogen gas concentration. H2S sensor 315 (U3) may be positioned on PCB 300 and may measure hydrogen sulfide gas concentration. VOC sensor 320 may be positioned on PCB 300 and may measure volatile organic compound concentrations. An AM component 325 (U4) and temperature/humidity sensor 330 (U5, H/T) may also be positioned on PCB 300. PCB 300 may further include a USB-C power connector 335, a resistor 340 (R1), and an LED indicator 345 (D1) to indicate operational status of patient unit 105.

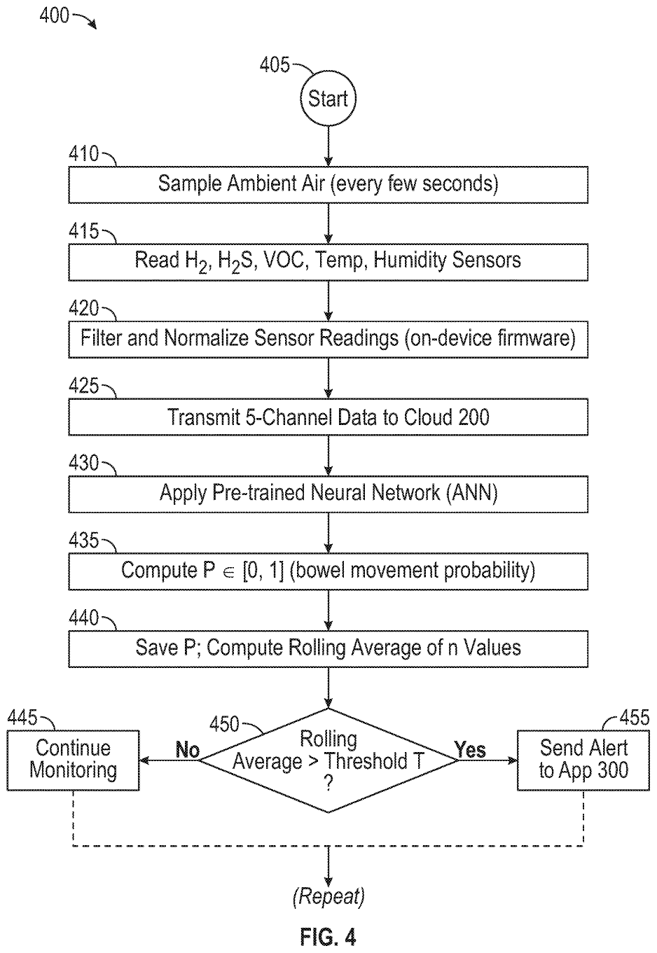

FIG. 4 illustrates a flowchart 400 of the event detection algorithm executed by cloud processing platform 110 in conjunction with patient unit 105. Beginning at start step 405, patient unit 105 may sample ambient air every few seconds at step 410, collecting readings from H2, H2S, VOC, temperature, and humidity sensors. At step 415, the raw sensor readings may be filtered and normalized on-device by firmware running on microcontroller 305. At step 420, the filtered and normalized five-channel sensor data may be transmitted to cloud processing platform 110 via Wi-Fi. At step 425, cloud processing platform 110 may apply a pre-trained artificial neural network (ANN) machine-learning model to the five-channel sensor data. At step 430, cloud processing platform 110 may compute probability P in the range [0, 1] representing the probability of a bowel movement. At step 435, cloud processing platform 110 may save P and compute a rolling average of n previous probability values. At decision step 445, cloud processing platform 110 may compare the rolling average to a predetermined threshold. If the rolling average does not exceed the threshold (450), the system continues monitoring (440). If the rolling average exceeds the threshold (455), cloud processing platform 110 may classify the event as a bowel movement and transmit an alert to mobile application 115. The rolling average approach may reduce false positive notifications caused by transient spikes in individual sensor readings.

The pre-trained ANN model may be trained on labeled sensor data collected from controlled exposures in which fecal material was placed near the sensor and baseline (no fecal material) conditions were recorded. Training data may be collected from multiple individuals to improve generalizability of the model across different individuals and room environments. The ANN may receive five inputs corresponding to the filtered and normalized readings from H2 sensor 310, H2S sensor 315, VOC sensor 320, temperature/humidity sensor 330 (temperature channel), and temperature/humidity sensor 330 (humidity channel), and may produce a single output P. The temperature and humidity inputs may serve as contextual features that allow the model to account for changes in ambient conditions that could affect gas sensor readings.

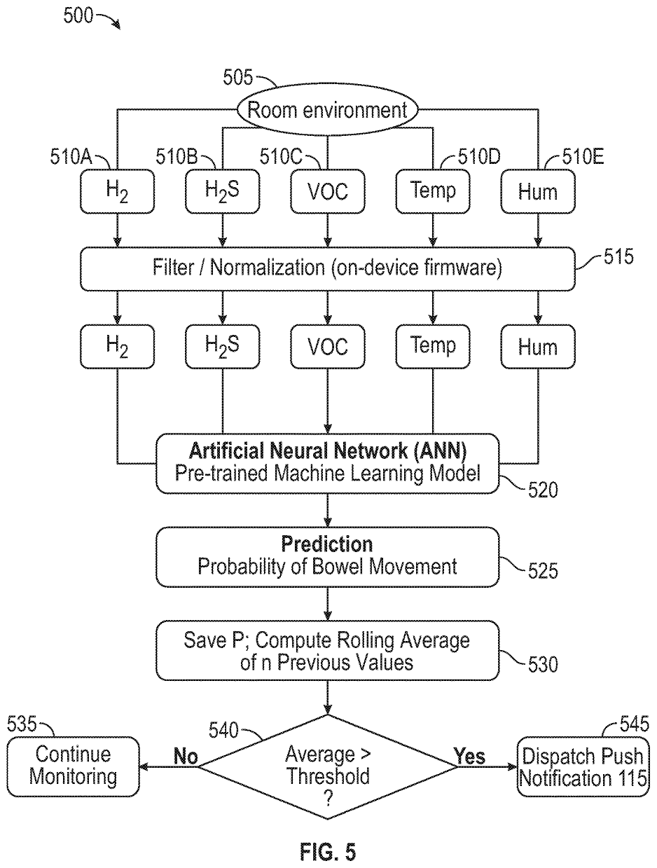

FIG. 5 illustrates a detailed data flow diagram 500 of the sensor signal processing pipeline. The five sensor input channels 505 may include H2 channel 510A, H2S channel 510B, VOC channel 510C, temperature channel 510D, and humidity channel 510E. These five channels may feed into a filter and normalization stage 515 implemented in on-device firmware on microcontroller 305. The filtered and normalized data may be input to a pre-trained artificial neural network (ANN) 520, corresponding to a pre-trained machine learning model (Pt ML M). The ANN 520 may output a prediction 525, specifically a bowel movement probability P (BM Probability). Cloud processing platform 110 may save P and compute a rolling average 530 of n previous probability values at step 535. At decision step 540, if the rolling average exceeds a predetermined threshold, cloud processing platform 110 may dispatch a push notification 545 to mobile application 115 on the caregiver's device 125. If the threshold is not exceeded, the system may continue monitoring.

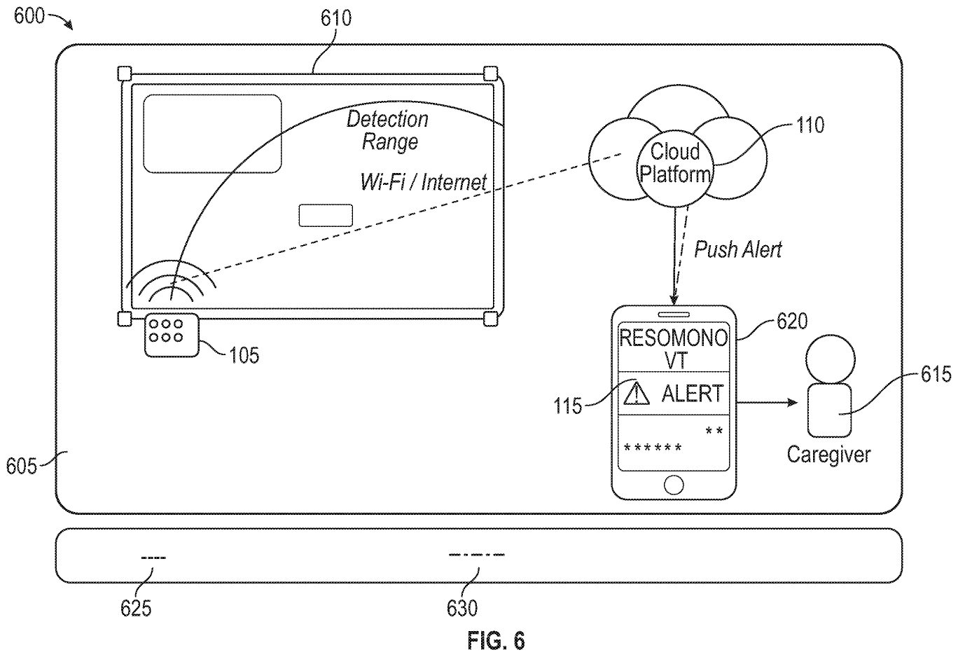

FIG. 6 illustrates a deployment diagram 600 showing an example installation of patient unit 605 in a room or environment near a dependent individual's bed. Patient unit 605 may detect chemical signatures of a bowel movement within a detection range 610 that is increased by the action of fan 210. Sensor data may be transmitted wirelessly via Wi-Fi and the internet 625 to cloud processing platform 110. When a bowel movement is detected, cloud processing platform 120 may send a push alert to mobile application 115 installed on a caregiver's mobile device 630, enabling the caregiver to respond promptly regardless of their physical location in the home or facility.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A bowel movement detection system, comprising:
a patient unit comprising:
a housing,
one or more gas sensors measuring concentrations of gases,
a wireless communication interface;
a cloud-based processing platform that receives sensor data from the patient unit over a network, applying a machine-learning model to the received sensor data to compute a bowel movement probability value,
transmitting an alert when the bowel movement probability value satisfies a threshold condition; and
a mobile application that receives the alert from the cloud-based processing platform and sends an alert to a device associated with a caregiver.

2. The system of claim 1, wherein the one or more gas sensors comprises:
a hydrogen gas sensor that measures hydrogen gas concentration.

3. The system of claim 1, wherein the one or more gas sensors comprises:
a hydrogen sulfide gas sensor that measures hydrogen sulfide gas concentration.

4. The system of claim 1, wherein one or more of the gas sensors comprises:
a volatile organic compound sensor that measures volatile organic compound concentrations.

5. The system of claim 1, wherein the patient unit further comprises a combined temperature humidity sensor that measures ambient temperature and ambient relative humidity, a hydrogen gas sensor, a hydrogen sulfide gas sensor, a volatile organic compound sensor, a temperature sensor, and a humidity sensor.

6. The system of claim 1, wherein the patient unit further comprises a fan that draws ambient air from outside the housing across the one or more gas sensors and expels the air through exhaust apertures in the housing.

7. The system of claim 1, wherein the patient unit further comprises a microcontroller that reads sensor outputs from the one or more gas sensors at periodic intervals, filters and normalizes the sensor outputs, and transmits the filtered and normalized sensor data to the cloud-based processing platform via the wireless communication interface.

8. The system of claim 5, wherein the wireless communication interface comprises a Wi-Fi antenna integrated with the microcontroller.

9. The system of claim 1, wherein the housing comprises air intake apertures on at least one side surface of the housing and air exhaust apertures on a top surface of the housing.

10. The system of claim 1, wherein the machine-learning model is a pre-trained artificial neural network that receives as input a vector comprising the sensor data and produces as output a scalar value between zero and one representing the bowel movement probability.

11. The system of claim 1, wherein the cloud-based processing platform maintains a rolling buffer of a plurality of consecutively computed bowel movement probability values, computes a rolling average of the plurality of values, and transmits the alert when the rolling average exceeds a predetermined threshold.

12. The system of claim 1, wherein the alert is a push notification transmitted to the mobile application on the device associated with a caregiver, and the mobile application further displays real-time sensor data and a history of bowel movement detection events.

13. A patient unit for a non-contact bowel movement detection system, the patient unit comprising:
a housing comprising air intake apertures on at least one side surface and air exhaust apertures on a top surface;
a microcontroller comprising a wireless communication interface;
a hydrogen gas sensor that measures a concentration of hydrogen gas in ambient room air;
a hydrogen sulfide gas sensor that measures a concentration of hydrogen sulfide gas in ambient room air;
a volatile organic compound sensor that measures concentrations of volatile organic compounds in ambient room air;
a combined temperature and humidity sensor;
a fan mounted within the housing that draws ambient air from outside the housing through the air intake apertures, across the sensors, and expels the air through the air exhaust apertures, thereby increasing a detection range of the patient unit;
wherein the microcontroller periodically reads outputs from the sensors and transmits sensor data to a remote processing platform via the wireless communication interface.

14. The patient unit of claim 13, wherein the microcontroller executes firmware that filters the sensor outputs to reduce noise, normalizes the filtered outputs to a common range, and transmits a five-element normalized sensor data vector to the remote processing platform.

15. The patient unit of claim 13, further comprising a power connector disposed on the housing that receives electrical power to operate the patient unit.

16. The patient unit of claim 13, further comprising an indicator that indicates an operational status of the patient unit.

17. The patient unit of claim 13, wherein the wireless communication interface comprises a Wi-Fi antenna integrated with the microcontroller.

18. The patient unit of claim 13, wherein the housing further comprises mounting features on a rear surface thereof that permit the patient unit to be mounted to at least one of: a flat surface, a bed post using a strap, and a wall using mounting apertures, thereby enabling flexible placement near a dependent individual.

19. The patient unit of claim 13, wherein the housing comprises air intake apertures distributed across at least two opposing side surfaces of the housing to promote uniform airflow across the sensors.

20. The patient unit of claim 13, wherein the fan draws ambient room air across the hydrogen gas sensor, the hydrogen sulfide gas sensor, the volatile organic compound sensor, and the combined temperature and humidity sensor, such that the patient unit passively and continuously monitors for bowel movement-associated chemical signatures without any action required by a dependent individual.
